(19) United States
(12) Patent Application Publication
(10) Pub. No.: US 2023/0226072 A1
(43) Pub. Date: Jul. 20, 2023

(54) METHYLENE BLUE AND RIBOFLAVIN FOR PROPHYLACTIC AND THERAPEUTIC ANTI-VIRAL THERAPY

(71) Applicant: Erich H. CERNY, Genf (CH)

(72) Inventors: Erich H. CERNY, Genf (CH); Caroline TAPPAREL VU, Chêne-Bougeries (CH); Thomas CERNY, St. Gallen (CH); Andreas CERNY, Lugano Ti (CH)

(21) Appl. No.: 18/010,346

(22) PCT Filed: Jun. 14, 2021
(86) PCT No.: PCT/EP2021/065888

§ 371 Date: Dec. 14, 2022

(30) Foreign Application Priority Data
Jun. 15, 2020 (CH) 00708/20
Feb. 26, 2021 (EP) 21159483.3

(51) Int. Cl. A61K 31/5415; A61K 31/525; A61P 31/16; A61P 31/14; A61K 9/00; A61K 9/20

(52) U.S. Cl. CPC A61K 31/5415; A61K 31/525; A61P 31/16; A61P 31/14; A61K 9/0053; A61K 9/2054; A61K 9/0043; A61K 9/0019

(57) ABSTRACT

Viral epidemic infections due to influenza, or coronaviruses, particularly influenza A and SARS-CoV-1 and -2 virus cause significant morbidity and mortality. The significant mutation rate of this group of viruses renders existing vaccines or antiviral drugs often useless, creating a need for broad acting, viral DNA or RNA sequence independent antiviral drugs. This invention describes the repurposing of methylene blue and riboflavin, two FDA approved drugs with an excellent safety profile. Using a virus neutralization assay of H1N1 Influenza A and SARS-CoV-2, the significant viricidal activity of both drugs at pharmacological dose and under physiological conditions is demonstrated. In vivo prophylactic and therapeutic use of the drugs for anti-viral application in an animal model is described.

### SUMMARY OF THE INVENTION

Viral epidemic infections due to influenza, or coronaviruses, particularly influenza A and SARS-CoV-1 and -2 virus cause significant morbidity and mortality. The significant mutation rate of this group of viruses renders existing vaccines or antiviral drugs often useless, creating a need for broad acting, viral DNA or RNA sequence independent antiviral drugs. This invention describes the repurposing of methylene blue and riboflavin, two FDA approved drugs with an excellent safety profile. Using a virus neutralization assay of H1N1 Influenza A and SARS-CoV-2, the significant viricidal activity of both drugs at pharmacological dose and under physiological conditions is demonstrated. In vivo prophylactic and therapeutic use of the drugs for anti-viral application in an animal model is described.

### BACKGROUND OF THE INVENTION

Wu W. et al., 2014, “Method of inactivating virus in circular blood and its applications in treating viral diseases”, U.S. Pat. No. 8,808,977, describes an in vitro procedure where methylene blue is added to blood. The blood is then recirculated with the help of a pump under a light source until the virus is fully neutralized. A removing device is used to absorb the photosensitizer before reinfusing the blood.

Floyd R. et al., “Antiviral therapy using thiazine dyes”, U.S. Pat. No. 6,346,529, describes an in vivo method for the treatment of human immunodeficiency virus (HIV) using thiazine dyes. This invention is limited to the application of a method of treatment to HIV and mentions methylene blue as a preferred embodiment.

Floyd R. et al., “Thiazine dyes used to inactivate HIV in biological fluids”, U.S. Pat. No. 5,827,644, describes an in vitro method using methylene blue and light to treat biological fluids against human immunodeficiency virus.

Zepp Ch. et al., “Method for inactivating non-enveloped viruses using a viricide-potentiating agent with a photoactivatable virucide”, U.S. Pat. No. 5,663,043, describes a method for inactivating in blood non enveloped viruses with the help of a photoactivatable virucide, and the administration of a viricide-potentiating chemical agent such as for example a cationic lipopolyamine.

Floyd R. et al., “Thiazine dyes used to inactivate HIV in biological fluids”, U.S. Pat. No. 5,571,666 describes a method for treating a biological fluid, obtained from a human for administration to a human patient in need thereof, in order to inactivate human immunodeficiency virus in the biological fluid comprising the steps of adding a thiazine dye and exposing the biological fluid to light.

Swartz M. R., “Method for inactivating viruses, bacteria, etc. in vitro and production of vaccines”, U.S. Pat. No. 4,402,318. A method of inactivating infectious agents in vitro is disclosed whereby methylene blue is activated by concurrent application of an electric field and light.

There are a number of publications, which use methylene blue and light, or Riboflavin and light to treat virus infected blood products in vitro. Neutralized viruses include SARS-CoV-1, SARS-CoV2, MERS and HIV:
Jin, C., et al., “Methylene blue photochemical treatment as a reliable SARS-COV-2 plasma virus inactivation method for blood safety and convalescent plasma therapy for the COVID-19 outbreak.” ResearchGate preprint, DOI: 10.21203/rs.3.rs-17718/v1, 2020.
Callahan, S. M., et al., Controlled inactivation of recombinant viruses with vitamin B2. J Virol Methods, 2008. 148(1-2): p. 132-45.
Eickmann, M., et al., Inactivation of Ebola virus and Middle East respiratory syndrome coronavirus in platelet concentrates and plasma by ultraviolet C light and methylene blue plus visible light, respectively. Transfusion, 2018. 58(9): p. 2202-2207.
Keil, S. D., et al., Inactivation of severe acute respiratory syndrome coronavirus 2 in plasma and platelet products using a riboflavin and ultraviolet light-based photochemical treatment. Vox Sang, 2020.
Xu, J., et al., Inactivation of SARS coronavirus in human plasma by methylene blue/light method. Jun shi yi xue ke xue yuan yuan kan=Bulletin of the Academy of Military Medical Sciences, 2005. 29(2): p. 142-144.

The prior art uses high energy light sources to activate methylene blue respectively riboflavin in order to generate in vitro a compound with viricidal activity.

The problem to be solved by the present invention is the provision of a therapeutic, in particular-immune response-independent, approach against viral epidemic infections due to influenza and/or coronaviruses, particularly influenza A and/or SARS-CoV-1 and -2 based on broad acting, viral DNA or RNA sequence independent, and therefore virus mutation independent, antiviral drugs.

### SUMMARY OF THE INVENTION

The above-mentioned problem could be solved by the surprising observation of the present inventors, that methylene blue and/or riboflavin, in order to generate virus-neutralizing activity, do not require high energy light sources.

Using unusual long incubation times, i.e. hours instead of minutes, between the drug and the virus in in vitro neutralization experiments, conditions simulating the in vivo situation were created. For the first time and unexpectedly a strong viricidal efficacy in the total dark (hermetically closed box) against Influenza N1H1 virus and SARS-CoV-2 could be demonstrated in vitro. This observation allows to use the two compounds in vivo, where only a limited quantity of light of the absorbance wave length of the drugs is transmitted through the skin of the individual receiving the treatment.

The present invention discloses methylene blue and riboflavin as broad acting, nucleic acid sequence independent antiviral compounds for prophylactic as well as therapeutic applications against viruses belonging to the Influenza and Coronavirus genera. Those viruses have a significant potential to mutate and it is therefore difficult to develop vaccines and to a certain degree also antiviral compounds covering future strains. The main mechanisms of said two molecules is based on corroding the nucleobase guanine as well as other viral molecules by singlet oxygen formation in vitro and in vivo. It is therefore reasonable to expect, that the two molecules will be viricidal against all influenza and coronaviruses in vivo. The energy required for the transfer of oxygen from its triplet ground state to the singlet excited state is relatively low (96 kJ/mol). In vivo conditions, i.e. normal body temperature and prolonged duration of treatment, are sufficient to generate significant levels of singlet oxygen.

The two drugs are the preferred embodiment for prophylactic and therapeutic application of this invention. Both molecules, methylene blue and riboflavin, show absence of known serious side effects even at very high dose and are FDA approved drugs. Methylene blue stains the urine, sclera and in white skinned probates also the skin lightly blue, but the effect is reversible after discontinuation of treatment. Riboflavin has no staining effect and may be preferred for a prophylactic application. Both compounds are FDA approved medications for use outside the field of virology.

The main targets among the aforementioned virus genera are Severe Acute Respiratory Syndrome coronavirus SARS-CoV-1 and SARS-CoV-2, which cause severe acute respiratory syndrome (SARS). The virus is an enveloped, positive-sense, single-stranded RNA virus entering the host cell by binding to the ACE2 receptor. Originating in 2003 and 2019, the virus provoked a worldwide pandemic.

Another equally important main target are Influenza viruses, particularly viruses belonging to Influenza A and there particularly the type N1H1, causing annual flue epidemics or pandemics with significant morbidity and mortality.

Under the condition of the right galenic formulation, dose, mode of application and application schedule, the two compounds deliver alone or in combination a surprisingly strong non virus sequence specific broad anti-viral activity (antiviral activity independent of specific viral amino acid or nucleotide sequences) as described in more detail below.

Both compounds can, depending on concentration and reaction partner, reduce or oxidize a compound. Methylene blue is able to take electrons on its aromatic thiazine ring to be reduced to leukomethylene blue (MBH₂) and transfer electrons to other compounds depending on the redox states and the concentration of methylene blue. Singlet oxygen is in a quantum state where all electrons are spin paired and corresponds to the lowest exited state of the diatomic oxygen molecule. Methylene blue as a sensitizer in combination with oxygen and a source of energy results in its production of singlet oxygen, a very reactive reaction partner which corrupts DNA or RNA by mechanisms such as guanine oxidation thereby having a broad non sequence specific viricidal activity. Further viricidal lesions include but are not limited to a) 8-oxo-7,8-dihydroguanine (8-oxoGua) lesions, b) modified carbonyl moieties on proteins, c) single-strand breaks (ssb) in the RNA genome d) RNA-protein crosslinks, all lesions correlating well with viricidal activity. Riboflavin has a similar mechanism of action based on oxygen singlet production.

### DESCRIPTION OF FIGURES

FIG. 1 demonstrates the viricidal efficacy of low dose methylene blue (0.25 mg/I) incubated with H1N1 influenza virus for 16 hours under white light (sterile hood, fluorescent lamp) and without white light (sterile hood, closed box), respectively.

FIG. 2 demonstrates the viricidal efficacy of a physiological dose of methylene blue (2.5 mg/I) incubated with SARS-CoV-2 virus for 20 hours under white light and without white light, respectively. Under these conditions there is a total viricidal effect (no bars) with methylene blue and methylene blue plus IgG even in the absence of light.

### DETAILED DESCRIPTION OF THE INVENTION

### (1) Materials, Terms, Definitions

Methylene Blue:

“Methylene blue” (MB) refers to a FDA approved drug, (NDA 204630) Compound CID: 6099, MF: C16H18CIN3S, MW: 319.9 g/mol InChlKey: CXKWCBBOMKCUKX-UHFFFAOYSA-M, IUPAC Name: [7-(dimethylamino)phenothiazin-3-ylidene]-dimethylazanium; chloride. Absorption max: 668, 609 nm (PubChem data base).

“Methylene blue Cation”: 3,7-Bis(dimethylamino)phenothiazin-5-ium; Methylthioninium; Compound CID: 4139, MF: C16H18N3S+ MW: 284.4 g/mol InChlKey: RBTBFTRPCNLSDE-UHFFFAOYSA-N, IUPAC Name: [7-(dimethylamino)phenothiazin-3-ylidene]-dimethylazanium.

Methylene blue is a redox dye, which means that depending on concentration and reaction partner it can reduce or oxidize a compound. More precisely, it is able to take electrons on its aromatic thiazine ring to be reduced to leukomethylene blue (MBH₂) and transfer electrons to other compounds depending on the redox states and the concentration of MB.

“Singlet oxygen” is oxygen in a quantum state where all electrons are spin paired corresponding to the lowest exited state of the diatomic oxygen molecule. A sensitizer (such as methylene blue or riboflavin) in combination with oxygen and a source of energy results in the production of singlet oxygen, a very reactive reaction partner which corrupts DNA or RNA by mechanisms such as guanine oxidation thereby having a broad non sequence specific viricidal activity: Observed chemical lesions (induced by singlet oxygen) in a Q beta phage systems include: a) 8-oxo-7,8-dihydroguanine (8-oxoGua) lesions, b) modified carbonyl moieties on proteins, c) single-strand breaks (ssb) in the RNA genome d) RNA-protein crosslinks, correlating well with viricidal effect measured. (Schneider, J. E., Jr., et al., Potential mechanisms of photodynamic inactivation of virus by methylene blue. I. RNA-protein crosslinks and other oxidative lesions in Q beta bacteriophage. Photochem Photobiol, 1998. 67(3): p. 350-7).

Other pharmacological effects of methylene blue found in the literature may contribute to its viricidal efficacy or have a clinical beneficial effect:

Distributive (hypovolemic) shock: Methylene blue produces a vasoconstriction in distributive shock by inhibition of nitric oxide synthase and guanylate cyclase. This is a concomitant and unexpected beneficial effect of methylene blue, because end stage viral infections present often the clinical status of a distributive shock. (Porizka, M., et al., Methylene blue administration in patients with refractory distributive shock—a retrospective study. Sci Rep, 2020. 10(1): p. 1828, Jang, D. H., L. S. Nelson, and R. S. Hoffman, Methylene blue for distributive shock: a potential new use of an old antidote. J Med Toxicol, 2013. 9(3): p. 242-9)

In Alzheimers Disease: Methylene blue oxidizes cysteine sulfhydryl groups on tau-protein to keep tau monomeric. One preclinical treatment study in tauopathy mice reported anti-inflammatory or neuroprotective effects mediated by the Nrf2/antioxidant response element (ARE); another reported insoluble tau reduction and a learning and memory benefit when given early.

In Methemoglobinemia: Methylene blue acts by reacting within red blood cells to form leukomethylene blue, which is a reducing agent of oxidized hemoglobin converting the ferric ion (Fe⁺⁺⁺) back to its oxygen-carrying ferrous state (Fe⁺⁺).

As antimalarial agent: Methylene blue, a specific inhibitor of P. falciparum glutathione reductase has the potential to reverse CQ (chloroquine) resistance and it prevents the polymerization of haem into haemozoin similar to 4-amino-quinoline antimalarials.

For ifosfamide induced neurotoxicity: Methylene blue functions as an alternate electron acceptor. It acts to reverse the NADH inhibition caused by gluconeogenesis in the liver while blocking the transformation of chloroethylamine into chloroacetaldehyde. In addition, it inhibits various amine oxidase activities, which also prevents the formation of chloroacetaldehyde.

Riboflavin:

“Riboflavin” or “Vitamin B2”, refers to a FDA approved drug, NDA 203324. Compound CID: 493570, MF: C17H20N4O6 MW: 376.4 g/mol, InChlKey: AUNGANRZJHBGPY-SCRDCRAPSAN, IUPAC Name: 7,8-dimethyl-10-[(2S,3S,4R)-2,3,4,5-tetrahydroxypentyl]benzo[g]pteridine-2,4-dione. UV max absorption: 220-225 nm, 266 nm, 371 nm, 444 nm, 475 nm . . . . Aqueous solutions are yellow showing a green fluorescence with max at 565 nm, O'Neil, M. J. (ed.). (The Merck Index —An Encyclopedia of Chemicals, Drugs, and Biologicals. Whitehouse Station, N.J.: Merck and Co., Inc., 2006., p. 1413).

Riboflavin is a precursor of the coenzymes flavin mononucleotide (FMN) and flavin adenine dinucleotide (FAD). These coenzymes are of vital importance in normal tissue respiration, pyridoxine activation, tryptophan to niacin conversion, fat, carbohydrate, and protein metabolism, and glutathione reductase mediated detoxification. In the context of the present invention riboflavin is a photosensitizer creating singlet oxygen, which denatures viral DNA and RNA (as explained above). We hypothesize the most important step for its viricidal activity consists in corroding guanine to its 8-oxo-7,8-dihydroguanine form, an effect based on singlet oxygen whereby photo activation augments the viricidal effect (as reported for MB, cf. above).

Epidemic, Pandemic:

The rapid expansion of a disease to a large number of people in a given population within a short period of time is an epidemic. Encompassing multiple countries or more one calls it a pandemic.

Influenza virus: Human influenza A, B, and C viruses cause seasonal epidemics, Influenza A is the typical pandemic flu virus. Influenza viruses contain seven or eight pieces of segmented negative-sense RNA. Influenza A viruses are divided into subtypes according to the hemagglutinin (H) and neuraminidase (N) on their surface. Methylene blue and riboflavin are viricidal based on a mechanism which is not virus sequence specific and the viricidal activity shown with the N1H1 strain is on theoretical grounds valid for all strains.

Coronavirus:

Coronaviruses have a capsid and contain a positive-sense, single-stranded RNA genome. There are 7 strains infecting humans, 3 of them may cause severe disease in humans: Middle East Respiratory Syndrome related coronavirus (MERS-CoV or MERS), Severe acute respiratory syndrome coronavirus 1 (SARS-CoV-1), Severe acute respiratory syndrome coronavirus 2 (SARS-CoV-2). SARS designates both of them. Methylene blue and riboflavin are viricidal based on a mechanism which is not virus sequence specific and the viricidal activity shown with the SARS-CoV-2 strain is on theoretical grounds valid for all corona strains.

Prophylactic Application and Therapeutic Application of an Antiviral Compound:

In the first case the probate is not yet infected and in the second case the probate is already infected by the virus. The probate population for prophylactic use concerns especially the population known to be at elevated risk for viral infection such as the medical personal treating virus infected people, persons with comorbidities (diabetes, leukemia, immunosuppression etc.) or people of advanced age. Absence of side effects is for a prophylactic application particularly important, because this is in most cases a healthy population. At prophylactic doses, riboflavin has no known side effects. Methylene blue colors urine blue and after a prolonged prophylaxis colors also the skin and sclera. This effect is fully reversible.

Virus Neutralization Assay, Plaque Forming Unit (PFU):

A plaque forming unit is a measure used in virology to describe the number of virus particles capable of forming plaques per unit volume, infecting a mono layer of susceptible cells. PFU is widely used as a measure for viral infectious efficacy in in vitro and in vivo experimentation.

For example, a solution of influenza virus with a concentration of 1,000 PFU/microliter indicates that 1 microliter of the solution contains enough virus particles to produce 1000 infectious plaques in a cell mono-layer. The counting of plaques in the monolayer of cells is today often replaced by immunological staining detecting the virus in infected cells with the help of enzyme labeled antibodies, therefore speeding up the procedure.

Viricidal Activity, Viricidal Efficacy:

This describes the pharmacological effect due to a compound, which diminishes the infectivity of the virus. The typical measure therefore in vitro is the counting of PFU in a virus neutralization assay and LD₅₀ or a clinical substitute such as elevated temperature, in in vivo testing. Counting of PFU is also possible in in vivo experiments: after infection, an organ can be homogenized and the PFU/weight ratio can be determined. Furthermore, the man skilled in the art uses generally accepted statistical methods to express LD₅₀ in PFU and vice versa.

Passive Immunization:

Historically, in passive immunization convalescence serum (i.e. blood serum that is obtained from an individual who has recovered from an infectious disease and contains antibodies against the infectious agent of the disease) or serum produced by active immunization containing neutralizing antibodies is given to a patient to protect against a disease. Convalescence serum can be replaced by neutralizing monoclonal antibodies directed against the virus. Methylene blue or riboflavin can be applied in combination with passive immunization. Methylene blue and riboflavin can be given concomitantly with poly- or monoclonal antibodies. It is know that antibodies have close to the binding side a catalytic site capable of producing singlet oxygen in the presence of water. (Datta, D., et al., Mechanism for antibody catalysis of the oxidation of water by singlet dioxygen. Proc Natl Acad Sci USA, 2002. 99(5): p. 2636-41. Wentworth, P., Jr., et al., Antibody catalysis of the oxidation of water. Science, 2001. 293(5536): p. 1806-11). The presence of singlet oxygen producing molecules such as methylene blue may potentiate this effect.

Light Source, Emitting Wave Length of a Light Source and Absorption Wave Length of Methylene Blue or Riboflavin:

The examples demonstrate an amplification of the viricidal effect of methylene blue and riboflavin in the presence of a light source emitting light within the absorption spectrum of the drugs. It is known and demonstrated in the examples, that energy transfer by light enhances the singlet oxygen production capacity of the drugs, leading to increased RNA and DNA damage. Light at the absorption maxima of methylene blue and riboflavin travels 4 to 5 mm under the skin, an area under the epidermis which is already well vascularized. It is therefore reasonable to assume that an external light source emitting light at the absorption wavelength of the drugs in vivo penetrates partially the skin and enhances the viricidal effect (Ash C, Dubec M, Donne K, Bashford T. Effect of wavelength and beam width on penetration in light-tissue interaction using computational methods. Lasers Med Sci. 2017; 32(8):1909-1918).

Applied Dose of Methylene Blue or Riboflavin:

The invention describes a compound containing methylene blue and/or riboflavin for the prophylaxis and/or treatment of influenza or corona virus infections of humans acting by its viricidal efficacy after application by the oral, intravenous, subcutaneous, intra muscular, intra nasal, rectal or nebulizer route through the nose or mouth, whereby the daily dose is not less than 10 microgram and not higher than 20 milligram per kg (i.e. 10 μg/kg to 20 mg/kg bodyweight of the patient per day) for the two compounds combined. The same dosage range applies if methylene blue and riboflavin are applied individually.

Virology Methods:

The man skilled in the art is familiar with the widely used virology methods described herein. The Virology Methods Manual, 1996, edited by Brian Mahy and Hillar Kangro is one of many comprehensive manuals for the methods to study, manipulate, and detect viruses. This text book completes the concise description of the methods and procedures given here and its content is herewith incorporated by reference.

Nebulizer:

A nebulizer is a drug delivery device used to administer medication in the form of a mist inhaled into the lungs. Classical applications are treatment of asthma, cystic fibrosis, COPD and other respiratory diseases or disorders. Nebulizers use oxygen, compressed air or ultrasonic power to break up solutions and suspensions into small aerosol droplets that are inhaled from the mouthpiece of the device. For example, a liquid methylene blue formula for injection as described herein, can be filled directly or with water diluted into the liquid reservoir of the nebulizer. The application with a nebulizer can be combined with oxygen administration and an oxygen mask. This type of application is particularly attractive in case of a viral lung infection.

### (2) Embodiments of the Invention

### (2.1) Particular Embodiments

The present invention relates to the following aspects and embodiments thereof:

According to a first aspect, the present invention relates to a compound or active ingredient selected from methylene blue, riboflavin and combinations thereof for use in the prophylactic and/or therapeutic treatment of an influenza and/or a corona virus infection of a human patient.

Methylene blue is known to minimize virus induced lung fibrosis, which is an additional valuable therapeutic effect associated with the present invention.

In particular, methylene blue, riboflavin and combinations of methylene blue and riboflavin are acting by their viricidal efficacy after application by the oral route, through intravenous, intra-nasal, subcutaneous or intra-muscular injection, by the rectal or nebulizer route or any combination thereof.

In a combination of methylene blue and riboflavin the constituents may be applied in any molar ratio. For example, the molar ratio of methylene blue to riboflavin may be in the range of 1:100 to 100:1, like 1:50 to 50:1, 1:20 to 20:1, 1:10: to 10:1 or 1:5 to 5:1, and preferably in about equimolar proportions of about 1:2 to about 2:1, like 1:1.

In a particular embodiment of said first aspect, the oral route of administration is applied for methylene blue.

In another particular embodiment of said first aspect, the oral route of administration is applied for riboflavin.

In still another particular embodiment of said first aspect, the oral route of administration is applied for a combination methylene blue and riboflavin.

In a particular embodiment of said first aspect, the intra-nasal route of administration is applied for methylene blue.

In another particular embodiment of said first aspect, the intra-nasal route of administration is applied for riboflavin.

In still another particular embodiment of said first aspect, the intra-nasal route of administration is applied for a combination methylene blue and riboflavin.

Intra-nasal application is particularly performed via administration of nasal sprays or nasal drops.

In the above embodiments of said first aspect the daily dose of said compound is in the range of 10 μg to 20 mg per kg bodyweight of the human patient.

More particularly the daily dose of methylene blue is in the range of 10 μg to 20 mg per kg bodyweight of the human patient; or the daily dose of riboflavin is in the range of 10 μg to 20 mg per kg bodyweight of the human patient; or the daily dose of a combination of methylene blue and riboflavin is in the range of 10 μg to 20 mg per kg bodyweight of the human patient

In the above embodiments of the first aspect the compound is used for prophylactic treatment.

Alternatively, in the above embodiments of the first aspect the compound is used for therapeutic treatment.

Alternatively, in the above embodiments of the first aspect the compound is used for prophylactic and therapeutic treatment.

In still another particular embodiment of said first aspect the compound is methylene blue administered orally, particularly in a daily dose of 0.1 to 10, more particularly 0.5 to 7.5, even more particularly 1 to 5, most particularly 3 to 5 mg/kg bodyweight of the patient.

Most particularly, the dose for viricidal treatment via the oral is 3 mg/kg in adults per 24 hours. The maximum dose should not exceed 5 mg/kg per day.

In still another particular embodiment of said first aspect the compound is methylene blue administered by injection, particularly in a daily dose of 0.1 to 10, more particularly 0.5 to 7.5, even more particularly 1 to 5, most particularly 2 to 4 mg/kg bodyweight of the patient.

Most particularly, the dose for viricidal treatment via injection is 3 mg/kg in adults per 24 hours. If given as a bolus it should be applied over a period of 5 at least minutes. The maximum dose should not exceed 5 mg/kg per day.

In still another particular embodiment of said first aspect the compound is methylene blue administered rectally, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is methylene blue administered via nebulizer, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is methylene blue administered intra-nasally, particularly in a daily dose of 0.1 to 10, more particularly 0.5 to 7.5, even more particularly 1 to 5, most particularly 2 to 4 mg/kg bodyweight of the patient

In still another particular embodiment of said first aspect the compound is methylene blue, administered by a combination of at least two routes of administration selected from the group of oral, intravenous, subcutaneous, intra-muscular, intra-nasal or nebulizer route, particularly in a combined daily dose of 0.1 to 10, more particularly 0.5 to 7.5, even more particularly 1 to 5, most particularly 2 to 4 mg/kg bodyweight of the patient.

In still another particular embodiment of said first aspect the compound is riboflavin administered orally, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is riboflavin administered by injection, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is riboflavin administered rectally, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is riboflavin administered via nebulizer, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is riboflavin administered intra-nasally, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is riboflavin, administered by a combination of at least two routes of administration selected from the group of oral, intravenous, subcutaneous, intra-muscular, intra-nasal or nebulizer route, particularly in a combined daily dose of 0.1 to 10, more particularly 0.5 to 7.5, even more particularly 1 to 5, most particularly 2 to 4 mg/kg bodyweight of the patient.

In still another particular embodiment of said first aspect the compound is a combination of methylene blue and riboflavin administered orally, particularly in a total combined daily dose of 0.1 to 10, more particularly 0.5 to 7.5, even more particularly 1 to 5, most particularly 2 to 4 mg/kg bodyweight of the patient.

In still another particular embodiment of said first aspect the compound is a combination of methylene blue and riboflavin administered by injection, particularly in a total combined daily dose of 0.1 to 10, more particularly 0.5 to 7.5, even more particularly 1 to 5, most particularly 2 to 4 mg/kg bodyweight of the patient.

In still another particular embodiment of said first aspect the compound is a combination of methylene blue and riboflavin administered rectally, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is a combination of methylene blue and riboflavin administered via nebulizer, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is a combination of methylene blue and riboflavin administered intra-nasally, particularly in a daily dose of 0.1 to 10, more particularly 0.5 to 7.5, even more particularly 1 to 5, most particularly 2 to 4 mg/kg bodyweight of the patient.

In still another particular embodiment of said first aspect the compound is a combination of methylene blue and riboflavin, administered by a combination of at least two routes of administration, either simultaneously or in any order, selected from the group of oral, intravenous, intra nasal, subcutaneous, intra muscular, or nebulizer route, particularly in a combined daily dose of 0.1 to 10, more particularly 0.5 to 7.5, even more particularly 1 to 5, most particularly 2 to 4 mg/kg bodyweight of the patient.

In still another particular embodiment of said first aspect, where the compound is a combination of methylene blue and riboflavin, methylene blue and riboflavin are administered separately or in admixture.

In still another particular embodiment of said first aspect, where the compound is a combination of methylene blue and riboflavin, methylene blue and riboflavin are administered separately via the same or different route of administration.

In still another particular embodiment of said first aspect, the influenza virus is an influenza A virus and the coronavirus is a SARS-CoV-1 or SARS-CoV-2 or MERS virus, more particularly, the influenza virus is an influenza A virus and the coronavirus is a SARS-CoV-2 virus.

In still another particular embodiment of said first aspect, methylene blue and/or riboflavin is formulated into a pharmaceutical composition, which allows for a slow release of methylene blue and/or riboflavin.

In still another particular embodiment of said first aspect, methylene blue, riboflavin or a combination thereof is applied to diminish fibrotic lung damage due to viral infection.

In still another particular embodiment of said first aspect, methylene blue, riboflavin or a combination thereof is applied in viral induced distributive shock acting on vasoconstriction of small vessels due to its effect on nitric oxide.

In still another particular embodiment of said first aspect, methylene blue, riboflavin or a combination thereof, is applied concomitantly with a further therapeutic agent, in particular monoclonal or polyclonal virus neutralizing antibodies, amplifying viricidal efficacy.

In still another particular embodiment of said first aspect, the compound is methylene blue and the influenza virus is an influenza A virus.

In still another particular embodiment of said first aspect, the compound is methylene blue and the coronavirus is a SARS-CoV-2 virus.

In still another particular embodiment of said first aspect, the compound is methylene blue in combination with riboflavin and the influenza virus is an influenza A virus and the coronavirus is a SARS-CoV-2 virus.

In still another particular embodiment of said first aspect, the compound is riboflavin and the influenza virus is an influenza A virus and the coronavirus is a SARS-CoV-2 virus.

In still another particular embodiment of said first aspect, the prophylactic treatment is performed until the epidemic virus exposition disappears.

In still another particular embodiment of said first aspect, the therapeutic treatment is performed over a period of 5 to 28 days, more particularly 5 to 14 days, even more particularly 5 to 10 days.

In still another particular embodiment of said first aspect, the treatment is performed in the absence of an external (extracorporal) high energy light source activating methylene blue.

According to a second aspect, the present invention provides a pharmaceutical orally applicable composition in solid or liquid form, comprising in a pharmaceutically acceptable carrier or diluent a viricidally effective amount of a compound selected from methylene blue, riboflavin or combinations thereof.

A particular embodiment of said second aspect relates to a liquid pharmaceutical composition for intra-nasal application of application via nebulizer,

Another particular embodiment of said second aspect relates to a pharmaceutical composition, comprising methylene blue, riboflavin or combinations thereof in a liquid pharmaceutically acceptable carrier in a proportion in the range of 0.1 to 2 wt.-%, particularly 0.5 to 1.5 wt.-%, and more particularly 0.8 to 1, 2 wt.-%, and especially about 1 wt.-%, based on the total weight of the composition.

Still another particular embodiment of said second aspect relates to a pharmaceutical composition formulated as nasal sprays or nasal drops.

According to a third aspect, the present invention provides a method for prophylactic and/ortherapeutic treatment of an influenza or corona virus infection of a human patient, which method comprises administering to the patient a viricidally effective amount of a compound selected from methylene blue, riboflavin and combinations thereof.

In particular, methylene blue, riboflavin or combinations thereof are applied in said method in the same manner as defined above the anyone of embodiments of the first aspect of the invention.

### (2.2) Further Embodiments

One or more compounds or “active agents” disclosed herein can be administered to a patient by themselves or in pharmaceutical compositions where they are mixed with biologically suitable carriers or excipient(s) at doses effective to prevent, treat, attenuate or ameliorate a disease or condition as described herein. Mixtures of these compounds can also be administered to the patient as a simple mixture or in suitable formulated pharmaceutical compositions.

“Patient” as used herein means human or non-human, in particular human, animals.

An “active agent” or “compound” in the context if the present invention means any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. All stereoisomers, diastereomers, Z- and E-forms, in purified and mixture forms are included. Accordingly, when a compound is recited by specific name or a class of compounds is recited, all these forms are intended to be included.

A “dosage form” is any unit of administration (“unit dose”) of one or more active agents as described herein.

The term “treating” or “treatment” refers to: (i) preventing a disease, disorder or condition from occurring in a patient which may be predisposed to the disease, disorder and/or condition but has not yet been diagnosed as having it; (ii) inhibiting the disease, disorder or condition, i.e., arresting its development; and (iii) relieving the disease, disorder or condition, i.e., causing regression of the disease, disorder and/or condition. In particular it encompasses a prophylactic or therapeutic treatment or combinations thereof.

The compounds or active ingredients of this invention are generally given as pharmaceutical compositions comprised of a therapeutically effective amount of at least one such compound or its pharmaceutically acceptable salt and a pharmaceutically acceptable carrier and may contain conventional excipients.

The phrase “pharmaceutically acceptable” is employed herein to refer to those compounds, materials, compositions, and/or dosage forms which are, within the scope of sound medical judgment, suitable for use in contact with the tissues of patients without excessive toxicity, irritation, allergic response, or other problem or complication commensurate with a reasonable risk/benefit ratio.

The invention includes all “pharmaceutically acceptable salt forms” of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

A “therapeutically effective amount” and/or “prophylactically effective amount” means an amount effective, when administered to a human or non-human patient, to provide any therapeutic and/or prophylactic benefit. More particularly, a “therapeutically effective amount” is an amount of a compound disclosed herein or a combination of two or more such compounds, which inhibits, totally or partially, the progression of the condition or alleviates, at least partially, one or more symptoms of the condition.

A therapeutic benefit may be an amelioration of symptoms of a diseased patient, e.g., an amount effective to decrease the symptoms of influenza and/or coronal viral infections of a diseased patient. I

In certain circumstances a patient may be at risk of a viral infection, but not yet infected. In this case a prophylactically effective amount of a compound is an amount sufficient to provide a significant positive effect on any symptoms of a disease, disorder or condition e.g. an amount sufficient to significantly reduce the frequency and severity of influenza and/or corona viral infection symptoms to occur.

A therapeutically effective amount can also be an amount, which is prophylactically effective. Prophylactic application and dosage schedules for infectious diseases are often reduced as compared to treatment dosage and schedules because the initial infectious load is inferior to the infectious load after infection and after replication.

“Frequency” of dosage may vary depending on the compound used and the particular type of infection treated. A dosage regimen of once per day is possible. Dosage regimens in which the active agent is administered for several times daily, as for example 2 to 10 times, like 2, 3, 4, 5, 6, 7, 8, 9 or 10 times may occasionally be more helpful.

It will be understood, however, that the specific dose level and frequency for any particular patient will depend upon a variety of factors including the activity of the specific compound employed, the age, body weight, general health, sex, diet, time of administration, route of administration, rate of excretion, drug combination and the severity of the particular disease in the patient undergoing therapy. Patients may generally be monitored for therapeutic or prophylactic effectiveness using assays suitable for the condition being treated or prevented, which will be familiar to those of ordinary skill in the art.

Solid compositions are normally formulated in dosage units and compositions providing from about 0.1 to 2000 mg of the active ingredient per dose are of interest. Some examples of dosages are 1 mg, 10 mg, 100 mg, 250 mg, 500 mg, and 1000 mg.

Liquid compositions are usually in dosage unit ranges. Generally, the liquid composition will be in a unit dosage range of 1-100 mg/mL. Some examples of dosages are 1 mg/mL, 10 mg/mL, 25 mg/mL, 50 mg/mL, and 100 mg/mL.

The invention also encompasses methods where the compound is given in combination therapy. That is, the compound can be used in conjunction with, but separately from, other agents useful in treating infection. In these combination methods, the compound will generally be given in a daily dose as specified above in conjunction with other agents. The other agents generally will be given in the amounts used therapeutically or prophylactically. The specific dosing regimen, however, will be determined by a physician using sound medical judgment.

For any compound or combination thereof used according to the present invention, the therapeutically and/or prophylactically effective dose can be estimated initially from cellular assays or animal models. For example, a dose can be formulated in cellular and animal models to achieve a circulating concentration range that includes the IC₅₀ as determined in cellular assays (i.e., the concentration of the test compound which achieves a half-maximal inhibition of a given activity). In some cases it is appropriate to determine the IC₅₀ in the presence of 3 to 5% serum albumin since such a determination approximates the binding effects of plasma protein on the compound. Such information can be used to more accurately determine useful doses in humans.

Toxicity and therapeutic efficacy of such compounds or combination thereof can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds or combinations thereof which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see e.g. Fingl et al., 1975, in “The Pharmacological Basis of Therapeutics”, Ch. 1 p. 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the desired effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen, which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The term “pharmaceutical composition” means a composition comprising at least one pharmaceutically active compound as described herein in combination with at least one additional pharmaceutical carrier, i.e., adjuvant, excipient or vehicle, such as diluents, preserving agents, fillers, stabilizers, extenders, binders, humidifiers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Ingredients listed in Remington's Pharmaceutical Sciences, 18th ed., Mack Publishing Company, Easton, Pa. (1999) for example, may be used.

A pharmaceutical composition as used herein may be presented in the form of a “dosage form” or “unit dose” and may comprise one or more active agents. Thus, a pharmaceutical composition as used herein could, for example, provide two active agents admixed together in a unit dose or provide two active agents combined in a dosage form wherein the active agents are physically separated and/or have different release rates.

A “combined pharmaceutical product” as used herein is a combination of two more doses of two or more different active agents combined in separate dosage forms which are not admixed.

Pharmaceutical compositions include any suitable “formulation” including, for example, capsules, tablets, injections and liquids and may be administered through any suitable route.

Suitable routes of administration may, for example, include oral, eyedrop, nasal drop, nasal spray, rectal, transmucosal, topical, or intestinal administration; parenteral delivery, including intramuscular, subcutaneous, intramedullary injections, as well as intrathecal, intravenous, intraperitoneal, intranasal, or intraocular applications.

Alternatively, one may administer the compound in a local rather than a systemic manner, for example, via injection of the compound directly into a subcutaneous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

For injection, and for intra-nasal use such as nasal drops, the agents of the invention may be formulated in aqueous solutions, preferably in physiologically compatible buffers such as Hanks' solution, Ringer's solution, or physiological saline buffer.

For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with or without the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds disclosed herein is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the cosolvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few hours up to over several days.

The pharmaceutical compositions may also comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound disclosed herein formulated in a compatible pharmaceutical carrier may also be prepared, placed in an appropriate container, and labelled for treatment of an indicated condition.

In some formulations it may be beneficial to use the compounds disclosed herein in the form of particles of very small size, for example as obtained by fluid energy milling.

The use of compounds disclosed herein in the manufacture of pharmaceutical compositions is illustrated by the following description. In this description the term “active compound” denotes any compound of the invention but particularly any compound which is the final product of one of the following nonlimiting examples.

a) Capsules

In the preparation of capsules, 100 parts by weight of active compound and 150 parts by weight of lactose can be de-aggregated and blended. The mixture can be filled into hard gelatin capsules, each capsule containing a unit dose or part of a unit dose of active compound.

b) Tablets

Tablets can be prepared, for example, from the following ingredients.

Parts by weight

| | |
|---|---|
| Active compound | 100 |
| Lactose | 150 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

C) ENTERIC COATED TABLETS

Tablets can be prepared by the method described in (b) above. The tablets can be enteric coated in a conventional manner using a solution of 20% cellulose acetate phthalate and 3% diethyl phthalate in ethanol:dichloromethane (1:1).

d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

The present invention will now be described in more detail by reference to the following nonlimiting, illustrative examples.

### EXPERIMENTAL PART

### Example 1: Methylene Blue, Galenic Formula for Different Routes of Application

a) Formula for Injection (Intravenous, Subcutaneous or Intra Muscular Application):

Methylthioninium chloride (methylene blue, chloride salt) is diluted in distilled water as a solution for intravenous injection at a concentration of 5 mg/ml. The ionic strength of pure water is adjusted with KCl, because chloride ions reduce the solubility. The pH value is adjusted to pH=4.5 because long term stability is compromised at higher pH. The solution is stored in dark glass ampoules of 5, 10 and 20 ml volume. Dark glass, because of methylene blue's light sensitivity. The solution is compatible with 5% glucose or 5% dextrose solution, but is not compatible with 0.9% saline solution due to the risk of precipitation.

The recommended dose for viricidal treatment is 2 mg/kg in adults per 24 hours. If given as a bolus it should be applied over a period of 5 at least minutes. The maximum dose should not exceed 5 mg/kg. Caution is advised in the case of impaired renal function. Sensitivity to thiazine dyes and G-6-PD deficiency are further contraindications. Pulse oximeters cannot be used.

b) Formula for Application by the Oral Route:

The formula for injection can also be taken by the oral route or as nasal drops. The bioavailability of methylene blue after oral administration is 72%, with peak plasma concentrations after two hours and an elimination half-life of 18 hours. Methylene blue's half-life in humans is five to 10 hours. The recommended dose per kg for 24 hours is identical to the dose recommended for injection. The excellent resorption of methylene blue after oral intake make oral intake an attractive choice.

Methylene blue can of course also be confected in dry form combined with a filler as a tablet or capsule.

c) Slow Release Formula for Application by the Oral Route:

300 mg methylene blue are confected as slow release tablets with 2.1 gram of a mixture composed of pharmaceutical glaze, rice bran, hydroxy-propyl methylcellulose, di-calcium phosphate, stearic acid, magnesium stearate, and silica. The peak plasma concentration is significantly prolonged.

d) Formula for Application with a Nebulizer:

A nebulizer is a drug delivery device used to administer medication in the form of a mist inhaled into the lungs. Classical applications are treatment of asthma, cystic fibrosis, COPD and other respiratory diseases or disorders. Nebulizers use oxygen, compressed air or ultrasonic power to break up solutions and suspensions into small aerosol droplets that are inhaled from the mouthpiece of the device or applied through an oxygen mask.

The liquid methylene blue formula for injection can be filled directly or with water diluted into the liquid reservoir of the nebulizer. The application with a nebulizer can be combined with oxygen administration and an oxygen mask. This type of application is particularly attractive in case of a viral lung infection. Methylene blue is known to minimize virus induced lung fibrosis, which is an additional valuable therapeutic effect. The nebulizer reservoir is filled with 0.1% methylene blue in physiological saline solution.

### Example 2: Riboflavin, Galenic Formula for Different Routes of Application

a) Formula for Oral Application:

Riboflavin is sold as an Over The Counter (OTC) drug as vitamin B2 by dozens of different vendors typically as 100 mg tablets at very competitive prices. Resorption of vitamin B2 per application is limited to about 27 mg at any one time from an oral dose given to an adult. Regular multiple intake is necessary to obtain a viricidal dose of 3 mg/kg per day for an adult. Alternatively and or in combination with multiple uptake, a slow release formula enhances uptake significantly. Close to nothing of the drug is stored in the liver, spleen, heart, and kidneys and excess riboflavin is excreted unchanged in the urine. The drug exhibits biphasic pharmacokinetics with initial and terminal half-lives of 1.4 and 14 h (D. B. McCormick, in Encyclopedia of Toxicology, Third Edition, 2014).

b) Formula for Injection (Intravenous, Subcutaneous or Intra Muscular Application):

Colored glass is mandatory for storage, because the flavins are sensitive to light if in solution. Riboflavin is water soluble (1 g dissolves in 3-15 L water, depending on the crystal structure) and slightly soluble in absolute alcohol (45 mg riboflavin dissolve in 1 l of absolute ethanol). Riboflavin is very soluble in dilute alkalies, but is unstable. Neutral and acidic solutions of riboflavin are stable in the dark but present a 3% decomposition per month at 27° C. at pH 6.

Vitamin B2 for intra muscular or intra venous application is commercially available at a concentration of typically 5 mg/ml in a water NaCl solution (e.g. Vitamin B2, Streuli Pharma AG, Switzerland).

c) Formula for Application with a Nebulizer:

As above for methylene blue. Riboflavin has the advantage of not coloring the skin or urine and a 0.1% solution in a pH neutral solution such as 0.9% NaCl can be used for application as nose drops or nasal spray.

### Example 3: In Vitro Viral Efficacy of Methylene Blue or Riboflavin Against Influenza N1H1 and SARS-CoV-2 Virus

Virus neutralization assay for Influenza-A H1N1, respectively SARS-CoV:

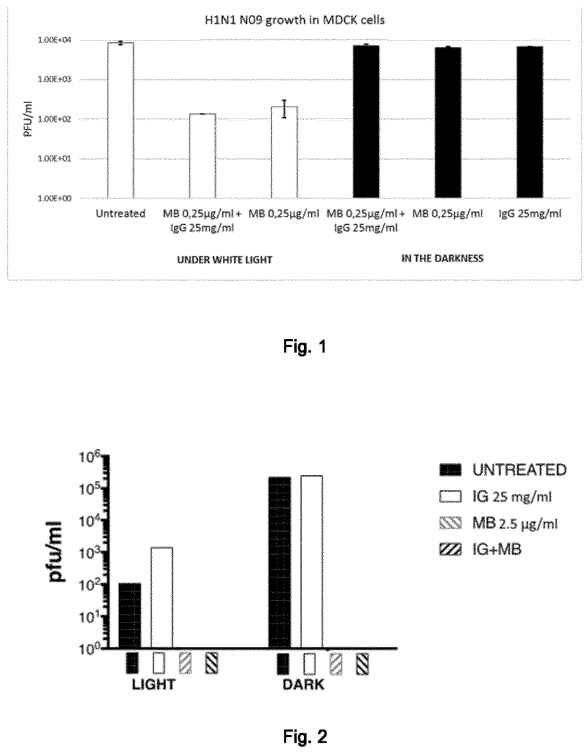

Upon incubation of the Influenza-A H1N1 virus with methylene blue as described below in the description of the figures in culture medium (the mix), the viral titer was determined on MDCK (Madin-Darby Canine Kidney Cells) as follows. MDCK cells were pre-plated 24 h in advance in 96-well plates. Serial dilutions of each mix were inoculated in duplicate on a confluent layer of cells for 2 h at 37° C. Following viral absorption, the viral inoculum (200 μl) was removed, the cells were washed and fresh DMEM (Dulbecco's Modified Eagle Medium)+GlutaMAX™ (Thermofisher catalog number 35050061), the culture medium, was added. After 24 h of incubation at 37° C., infected cells were detected by immunocytochemical (ICC) assay, using a mouse mAb Influenza A Antibody (Light Diagnostics) and an anti-mouse HRP conjugated antibody and the staining was visualized using the DAB substrate (FIG. 1).

For SARS-CoV-2 Vero-E6 cells were pre-plated 24 h in advance in 96-well plates. Serial dilutions of each mix were inoculated in duplicate with 1:10 dilutions in 180 μl on a confluent layer of cells for 2 h at 37° C. Following viral absorption, the viral inoculum (200 μl) was removed, the cells were washed and were overlaid with DMEM+GlutaMAX™ supplemented with 1.2% Avicel 581 (Dupont Pharma) and 5% FBS (Fetal Bovine Serum). After 72 h of incubation the plates were fixed with paraformaldehyde 4%, stained with crystal violet and the wells were scored according to presence or absence of cytopathic effect and the titer calculated with the TCI D50 method.

For the light exposure, the cold cathode fluorescent light (220W) of the sterile hood was used. PRIVIGEN sol perf 2.5 g/25 ml i. v. (CSL Behring AG) was used as a source of human immunoglobulin (IG). This serum does not contain anti-viral antibodies. Statistical analysis was done with Prism software (Prism 8, GraphPad). Experiments performed at a BSL-3, government approved facility.

Results with riboflavin are not shown but a similar efficacy is expected.

For the following in vitro experiments, the incubation time of the virus with the antiviral drug is particularly long, 16 hours for FIGS. 1 and 20 hours for FIG. 2. This allowed to put in evidence the strong antiviral efficacy of the drug also in the absence of light.

FIG. 1 demonstrates the viricidal efficacy of low dose methylene blue (0.25 mg/I) incubated with H1N1 influenza virus for 16 hours under white light (sterile hood, fluorescent lamp) and without white light (sterile hood, closed box), respectively. The y-axis shows log PFU units, the x-axis different experimental conditions. It is important to keep in mind that bar height and viricidal efficacy are inversely proportional. An absence of a bar means no detectable virus and therefore maximal viricidal efficacy. A bar at maximal height means no attenuation of PFU and therefore absence of viricidal effect.

FIG. 1 shows no viricidal effect in the absence of light. The very discrete diminution in viricidal effect in the presence of light and non-virus specific immunoglobulin is very probably due to nonspecific adsorption of the charged methylene blue molecule to immunoglobulin.

FIG. 2 demonstrates the viricidal efficacy of a physiological dose methylene blue (2.5 mg/I) incubated with SARS-CoV-2 virus for 20 hours under white light and without white light, respectively. Under these conditions there is a total viricidal effect (no bars) with methylene blue and methylene blue plus IgG even in the absence of light.

The pseudo moderate viricidal efficacy in the presence of light in the control without immunoglobulin and control with immunoglobulin is due to the effect of increased temperature under the sterile hood due to the light source after 20 hours of incubation.

Results observed with riboflavin provide evidence fora similar efficacy like methylene blue.

### Example 4: In Vivo Antiviral Efficacy of Methylene Blue and Riboflavin

Prophylaxis and therapeutic treatment of Balb/c mice from Influenza N1H1 infection with methylene blue:

For all mice except controls: Methylene blue (MB, C16H18CIN3S*3 H2O, Sigma) was administered via the drinking water supplemented with saccharin (1 tablet per 200 ml). Mice received a daily MB-dose of 5 mg/kg based on a daily drinking volume of ˜5-6 ml and a body weight of 25-35 g. The control group received drinking water with saccharin but without methylene blue.

a) Prophylactic Treatment with Methylene Blue, (Treatment Before Viral Challenge):

Groups of 6 female Balb/c mice (8-10 weeks old) were given methylene blue in drinking water on day 0 (day 0) and challenged intranasally with 1*10⁵ TCID₅₀ per animal of Influenza A N1H1 on day 10 and lungs were removed three days later and frozen at ≤60° C. for virus titer determination.

The control group of 6 female Balb/c mice (8-10 weeks old) received the same treatment, but the drinking water did not contain methylene blue.

b) Therapeutic Treatment with Methylene Blue (Treatment after Viral Challenge):

Groups of 6 female Balb/c mice (8-10 weeks old) were given methylene blue in drinking water on day 11 (day 11) and challenged intranasally with 1*10⁵ TCID₅₀ per animal of Influenza A N1H1 on day 10 and lungs were removed three days later and frozen at 60° C. for virus titer determination.

The control group of 6 female Balb/c mice (8-10 weeks old) received the same treatment, but the drinking water did not contain methylene blue.

c) Preparation of the Lung Samples for Virus Titration:

Mice were euthanized and lungs were removed on day 3 post challenge with influenza H1N1 virus. These tissue samples were stored at <−60° C. until they were transferred into homogenization tubes containing 1 ml cell medium supplemented with antibiotics. The lungs were homogenized two times at 5000 rpm for twenty seconds with 10 seconds pause between the intervals with a tissue homogenizer. The infectious H1N1 virus titer in homogenized lung samples was determined by a TCID₅₀ assay performed by titration on Madin-Darby Canine Kidney (MDCK) cells as described above.

The results of the TCID₅₀ assay demonstrate a significant viricidal efficacy of methylene blue in the case of prophylactic and therapeutic application. Analogous results are expected if methylene blue is replaced by riboflavin.

Further embodiments of the invention are:
1) A compound containing methylene blue and/or riboflavin for the prophylaxis and/or treatment of influenza or corona virus infections of humans acting by its viricidal efficacy after application by the oral, intravenous, subcutaneous, intra muscular, rectal or nebulizer route, whereby the daily dose is not less than 10 microgram and not higher than 20 milligram per kg for the two compounds combined.
2) The compound of embodiment 1, wherein the influenza virus is an influenza A virus and the coronavirus is a SARS-CoV-1 or SARS-CoV-2 or MERS virus.
3) The compound of embodiment 1, wherein the compound allows for a slow release of methylene blue and/or riboflavin.
4) The compound of embodiment 1, which is applied to diminish fibrotic lung damage due to viral infection.
5) The compound of embodiment 1, which is applied in viral induced distributive shock acting on vasoconstriction of small vessels due to its effect on nitric oxide.
6) The compound of embodiment 1 being applied concomitantly with monoclonal or polyclonal virus neutralizing antibodies amplifying viricidal efficacy.
7) The compound of embodiment 1, being activated in vivo through an external light source with an emission spectrum covering at least in part the absorption spectrum of methylene blue and or riboflavin.
8) The light source of embodiment 7, having an emission spectrum in accordance with the absorption spectrum of methylene blue and/or riboflavin.

1. A compound selected from methylene blue, riboflavin and combinations thereof for use in the prophylactic and/or therapeutic treatment of an influenza and/or a corona virus infection of a human patient.

2. The compound for use of claim 1, acting by its viricidal efficacy after application by the oral route, intra-nasal application, through intravenous, subcutaneous or intra muscular injection, by the rectal or nebulizer route or any combination thereof, in particular by the oral route.

3. The compound for use of claim 1 or 2, whereby the daily dose of said compound is in the range of 10 μg to 20 mg per kg bodyweight of the human patient.

4. The compound for use of anyone of the claims 1 to 3, which is methylene blue.

5. The compound for use of anyone of the claims 1 to 3, which is riboflavin.

6. The compound for use of anyone of the claims 1 to 3, which is a combination of methylene blue and riboflavin.

7. The compound for use of anyone of the claims 1 to 6, which is used for prophylactic treatment.

8. The compound for use of anyone of the claims 1 to 6, which is used for therapeutic treatment.

9. The compound for use of anyone of the claims 1 to 6, which is used for prophylaxis and therapeutic treatment.

10. The compound for use of claim 4, which is methylene blue administered orally, particularly in a daily dose of 0.1 to 10, more particularly 0.5 to 7.5, even more particularly 1 to 5, most particularly 2 to 4 mg/kg bodyweight of the patient.

11. The compound for use of claim 4, which is methylene blue administered by injection, particularly in a daily dose of 0.1 to 10, more particularly 0.5 to 7.5, even more particularly 1 to 5, most particularly 2 to 4 mg/kg bodyweight of the patient.

12. The compound for use of claim 4, which is methylene blue administered rectally, particularly in a daily dose of 0.1 to 10, more particularly 0.5 to 7.5, even more particularly 1 to 5, most particularly 2 to 4 mg/kg bodyweight of the patient.

13. The compound for use of claim 4, which is methylene blue administered intra-nasally or via nebulizer or a combination thereof, particularly in a daily dose of 0.1 to 10, more particularly 0.5 to 7.5, even more particularly 1 to 5, most particularly 2 to 4 mg/kg bodyweight of the patient.

14. The compound for use of claim 4, which is methylene blue, administered by a combination of at least two routes of administration selected from the group of oral, intravenous, subcutaneous, intra muscular, intra-nasal or nebulizer route, particularly in a combined daily dose of 0.1 to 10, more particularly 0.5 to 7.5, even more particularly 1 to 5, most particularly 2 to 4 mg/kg bodyweight of the patient.

15. The compound for use of claim 5, which is riboflavin administered orally, particularly in a daily dose of 0.1 to 10, more particularly 0.5 to 7.5, even more particularly 1 to 5, most particularly 2 to 4 mg/kg bodyweight of the patient.

16. The compound for use of claim 5, which is riboflavin administered by injection, particularly in a daily dose of 0.1 to 10, more particularly 0.5 to 7.5, even more particularly 1 to 5, most particularly 2 to 4 mg/kg bodyweight of the patient.

17. The compound for use of claim 5, which is riboflavin administered rectally, particularly in a daily dose of 0.1 to 10, more particularly 0.5 to 7.5, even more particularly 1 to 5, most particularly 2 to 4 mg/kg bodyweight of the patient.

18. The compound for use of claim 5, which is riboflavin administered intra-nasally or via nebulizer or a combination thereof, particularly in a daily dose of 0.1 to 10, more particularly 0.5 to 7.5, even more particularly 1 to 5, most particularly 2 to 4 mg/kg bodyweight of the patient.

19. The compound for use of claim 5, which is riboflavin, administered by a combination of at least two routes of administration selected from the group of oral, intravenous, subcutaneous, intra muscular, intra-nasal or nebulizer route, particularly in a combined daily dose of 0.1 to 10, more particularly 0.5 to 7.5, even more particularly 1 to 5, most particularly 2 to 4 mg/kg bodyweight of the patient.

20. The compound for use of claim 6, which is a combination of methylene blue and riboflavin administered orally, particularly in a total combined daily dose of 0.1 to 10, more particularly 0.5 to 7.5, even more particularly 1 to 5, most particularly 2 to 4 mg/kg bodyweight of the patient.

21. The compound for use of claim 6, which is a combination of methylene blue and riboflavin administered by injection, particularly in a total combined daily dose of 0.1 to 10, more particularly 0.5 to 7.5, even more particularly 1 to 5, most particularly 2 to 4 mg/kg bodyweight of the patient.

22. The compound for use of claim 6, which is a combination of methylene blue and riboflavin administered rectally, particularly in a daily dose of 0.1 to 10, more particularly 0.5 to 7.5, even more particularly 1 to 5, most particularly 2 to 4 mg/kg bodyweight of the patient.

23. The compound for use of claim 6, which is a combination of methylene blue and riboflavin administered intra-nasally or via nebulizer or a combination thereof, particularly in a daily dose of 0.1 to 10, more particularly 0.5 to 7.5, even more particularly 1 to 5, most particularly 2 to 4 mg/kg bodyweight of the patient.

24. The compound for use of claim 6, which is a combination of methylene blue and riboflavin, administered by a combination of at least two routes of administration (simultaneously or in any order) selected from the group of oral, intravenous, subcutaneous, intra muscular, intra-nasal or nebulizer route, particularly in a combined daily dose of 0.1 to 10, more particularly 0.5 to 7.5, even more particularly 1 to 5, most particularly 2 to 4 mg/kg bodyweight of the patient.

25. The compound for use according to anyone of the claims 20 to 24, wherein methylene blue and riboflavin are administered separately or in admixture.

26. The compound for use according to claim 24, wherein methylene blue and riboflavin are administered separately via the same or different route of administration.

27. The compound for use of anyone of the claims 1 to 26, wherein the influenza virus is an influenza A virus and the coronavirus is a SARS-CoV-1 or SARS-CoV-2 or MERS virus.

28. The compound for use of anyone of the claims 1 to 26, wherein the influenza virus is an influenza A virus and the coronavirus is a SARS-CoV-2 virus.

29. The compound for use of anyone of the claims 1 to 26, wherein the compound is formulated into a pharmaceutical composition, which allows for a slow release of methylene blue and/or riboflavin.

30. The compound for use of anyone of the claims 1 to 26, which is applied to diminish fibrotic lung damage due to viral infection.

31. The compound for use of anyone of the claims 1 to 26, which is applied in viral infection-induced distributive shock acting on vasoconstriction of small vessels due to its effect on nitric oxide.

32. The compound for use of anyone of the claims 1 to 26, being applied concomitantly with a further therapeutic agent, in particular monoclonal or polyclonal virus neutralizing antibodies, amplifying viricidal efficacy.

33. The compound for use of anyone of the claims 1 to 26, wherein the compound is methylene blue and the influenza virus is an influenza A virus.

34. The compound for use of anyone of the claims 1 to 26, wherein the compound is methylene blue and the coronavirus is a SARS-CoV-2 virus.

35. The compound for use of anyone of the claims 1 to 26, wherein the compound is methylene blue in combination with riboflavin and the influenza virus is an influenza A virus and the coronavirus is a SARS-CoV-2 virus.

36. The compound for use of anyone of the claims 1 to 26, wherein the compound is riboflavin and the influenza virus is an influenza A virus and the coronavirus is a SARS-CoV-2 virus.

37. The compound for prophylactic use according to anyone of the claims 1 to 36, wherein the treatment is performed until the epidemic virus exposition disappears.

38. The compound for therapeutic use according to anyone of the claims 1 to 36, wherein the treatment is performed over a period of 5 to 28 days, more particularly 5 to 14 days, even more particularly 5 to 10 days, most particularly 5 days.

39. The compound for use according to anyone of the claims 1 to 38, wherein the treatment is performed in the absence of an external (extracorporal) high energy light source activating methylene blue.

40. A pharmaceutical applicable composition in solid or liquid form, comprising in a pharmaceutically acceptable carrier or diluent a viricidally effective amount of a compound selected from methylene blue, riboflavin or combinations thereof, in particular a liquid composition for intra-nasal application of application via nebulizer.

41. The pharmaceutical composition of claim 40, comprising methylene blue, riboflavin or combinations thereof in a liquid pharmaceutically acceptable carrier in a proportion in the range of 0.1 to 2 wt.-%, particularly 0.5 to 1.5 wt.-%, and more particularly 0.8 to 1, 2 wt. %, and especially about 1 wt.-%, based on the total weight of the composition.

42. The composition of claim 40 or 41 formulated as nasal sprays or nasal drops.

43. A method for prophylaxis and/or treatment of an influenza or corona virus infection of a human patient, which method comprises administering to the patient a viricidally effective amount of a compound selected from methylene blue, riboflavin and combinations thereof.

44. The method of claim 43, wherein the compound is applied in said method as defined in anyone of the claims 1 to 39.
